(19) United States
(12) Patent Application Publication
(10) Pub. No.: US 2019/0021960 A1
(43) Pub. Date: Jan. 24, 2019

(54) PACKAGING-COMPATIBLE AEROSOL SHOWER FOAM

(71) Applicant: BEIERSDORF AG, Hamburg (DE)

(72) Inventors: Rixa DIPPE, Hamburg (DE); Romy KIENAST, Hamburg (DE); Kirsten SCHNEIDER, Ahrensburg (DE); Ina SCHORNSTEIN, Pinneberg (DE); Britta FISCHER, Wedel (DE)

(21) Appl. No.: 16/081,354

(22) PCT Filed: Feb. 13, 2017
(86) PCT No.: PCT/EP2017/053104

§ 371 Date: Aug. 30, 2018

(30) Foreign Application Priority Data
Mar. 2, 2016 (DE) 10 2016 203 440.5

(51) Int. Cl. A61K 8/04; A61Q 19/10

(52) U.S. Cl. CPC A61K 8/046; A61K 2800/87; A61K 2800/30; A61Q 19/10

(57) ABSTRACT

Aerosol shower foam preparations that are suitably mild, have a creamy dense foam and are packaging-compatible.

The present invention describes cosmetic aerosol shower foam preparations which are notable for good mildness, a creamy, thick foam, and packaging compatibility.

Cleansing oneself has been a need for humans for millennia. Cleansing is intended to remove dirt particles from the skin and then rinse them away. In order to achieve this, there are already many various forms of cleansing composition. Since cleansing does not only remove dirt particles from the skin, but also the skin's own lipids and water-binding substances (natural moisturizing factors, NMF) are always removed and rinsed away alongside them, consumers have a great desire to use cleansing products that are as mild as possible in order for this not to occur to such a great extent and therefore for the skin not to be so heavily stressed. Stressing of the skin may be reflected in the form of dry, brittle skin, reddened skin and/or tight skin. These phenomena gain even more significance against the background of many people taking daily showers or baths, when appropriate, even many times per day. This cleansing behavior makes it particularly important for the products used to be mild, so that the skin is stressed as little as possible.

Alongside the consumers' basic need for cleansing, there is an increasing desire to be pampered during body cleansing, and to treat oneself to the “little luxuries” of the bathroom. Part of this, for example, is that the products are notable for a luxurious product form, and that the use is associated with pleasant sensory impressions. One possibility for eliciting these pleasant sensory impressions consists in the feel of the products, when dispensing them and applying them to the skin, causing pleasant sensations. This is for example achieved by a foam which is notable for its creaminess and fine bubbles.

In order for the product form to be perceived as luxurious, it should differ and stand out from the currently conventional product form of shower preparations in plastic bottles. This can for example be achieved with aerosol foams, which are advantageously provided in cans.

Therefore, it is an object of the present invention to provide mild cleansing preparations in a product form which meets the consumers' desire for special, luxurious products.

Surprisingly, mild cleansing preparations with a creamy, finely-bubbled foam were able to be provided by these preparations being incorporated into aerosol containers as foamable and/or post-foaming preparations.

A large number of cleansing products are known which use various possibilities in order to provide mild preparations for the skin. For example, mention may be made here of EP 1430884 B1, which discloses a shower oil comprising a mild surfactant mixture and oily substances.

Aerosol foams in aerosol containers are known per se and are commonly encountered in the cosmetic sectors relating to hair preparations, shaving foams or sun-protection preparations in foam form. For example, mention may be made of EP 1374837 B1. This document discloses aerosol foam products for treating the hair, which comprise specific polymers and a solvent system and are decanted into a pressure-resistant packaging together with propellants.

Even older documents of the prior art also disclose preparations which are post-foaming and are offered in pressurized gas containers. Document U.S. Pat. No. 3,541,581 names, as essential constituents of such a composition, water, soap (water-soluble salts of higher fatty acids), gelling agents and post-foaming agents.

Document U.S. Pat. No. 4,405,489 proposes dispensing with a gelling agent, but in this case a specific and expensive process for preparing and decanting such compositions is required.

Document DE 3839349 A1 discloses low-viscosity, surfactant-containing cleansing solutions which are converted into a cleansing gel by application of pressure with highly volatile gases.

As can be seen from the prior art, the production process of such preparations is in some cases extremely expensive. Nevertheless, there is still a need to prepare such preparations, since they accommodate the consumers' desires for special, even luxurious, products.

It has transpired that mild cosmetic cleansing preparations can be provided if the surfactant content does not exceed 10 wt % based on the active content. In order to achieve a creamy, finely-bubbled foam in such preparations, the cleansing preparations are filled as aerosol foams with a propellant into aerosol containers.

It has however become apparent that the aerosol containers are attacked by the cleansing preparations; corrosion phenomena appear. Even the use of aerosol containers with inner protective coating does not prevent this. The inner protective coating is infiltrated by the cosmetic cleansing preparations, becomes detached and corrosion occurs. Corrosion of aerosol containers can lead to leaking of the container and bursting of the container. The detachment of the inner protective coating can furthermore lead to blockage of the valve, and as a result the product becomes unusable.

The resulting object is that of providing mild cleansing preparations, which form a creamy and finely-bubbled foam and can be provided in aerosol containers, without the inner protective coating of the aerosol container being damaged.

Surprisingly, the object was achieved by cosmetic cleansing preparations comprising
≤0.5 wt % NaCI, based on the cleansing preparation,
≤10 wt % surfactant or surfactants, based on the active content and the cleansing
   preparation, selected from the group of anionic, amphoteric and nonionic surfactants,

wherein the cosmetic cleansing preparations comprise 0 to 1.5 wt % of uncharged organic molecules having a molar mass of <250 g/mol, and

wherein the cleansing preparations and a propellant are filled into aerosol containers with an inner protective coating.

It is preferable if the inventive cleansing preparations are free of soaps. In the context of the present invention, soaps are intended to mean the water-soluble salts of higher fatty acids. In the context of the present invention, “free of” means that there is less than 0.1 wt % of the respective substance present, based on the weight of the cleansing preparation. If traces of soap are present in the inventive preparations, they originate from impurities in the constituents or premixes which are used to prepare the preparations.

It is essential to the invention that the indication of ≤10 wt % surfactant or surfactants always relates to the total amount of surfactant or surfactants in the cleansing preparation.

It is preferable if the anionic surfactant(s) is/are selected from the group of sodium laureth sulfate, disodium laureth sulfosuccinate and/or sodium cocosulfate. The anionic surfactant or the anionic surfactants are present at a content of 1 to 10 wt %, preferably 3 to 7 wt %. If the content of the anionic surfactant(s) is 10 wt %, then there are no further surfactants contained in the cosmetic preparations. If there are further surfactants present alongside an/the anionic surfactant(s), for example amphoteric and/or nonionic surfactants, then the total amount of surfactants must not exceed a value of 10 wt %.

Preferred amphoteric surfactants are low-salt forms of alkyl amphoacetates, such as disodium cocoamphodiacetate, disodium cocoamphomonoacetate. Preferred embodiments are entirely without amphoteric surfactants.

Advantageous nonionic surfactants may be selected from the group of alcohols, cocamides, such as MEA, DEA or MIPA, esters arising from the esterification of carboxylic acids with ethylene oxide, glycerol, sorbitan or other alcohols, PEG-200 Hydrogenated Glyceryl Palmate, ethoxylated and/or propoxylated triglyceride esters, propoxylated POE ethers and alkyl polyglycosides such as lauryl glucoside, decyl glycoside and coco glycoside, sucrose esters, sucrose ethers, polygycerol esters, diglycerol esters, monoglycerol esters, methylglucose esters and esters of hydroxy acids.

Preference is given to surfactant combinations of large surfactant molecules, because large surfactant molecules do not interact with the inner protective coating. One advantageous surfactant combination consists of sodium laureth sulfate and disodium laureth sulfosuccinate, in a preferred weight-ratio in the range from 2:1 to 1:2.

It is preferable if the content of all surfactants contained in the cleansing preparation is selected from a range from 1 to 10 wt %, preferably 2 to 8 wt %, based on the weight of the cleansing preparation.

In one advantageous embodiment, only one or a plurality of anionic surfactant(s) are present in the cleansing preparation.

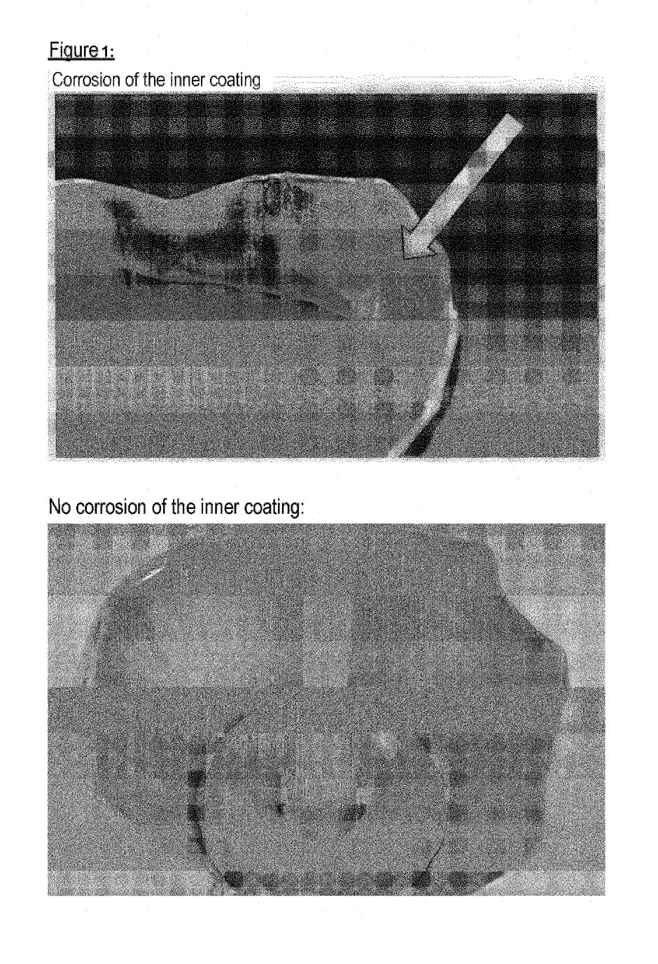

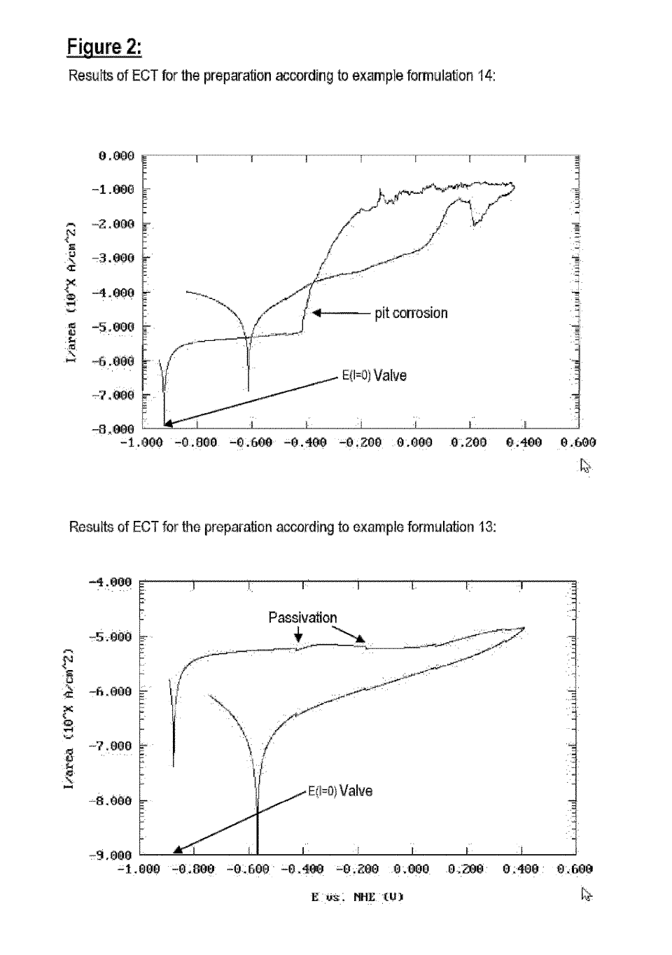

It is essential to the invention that the cosmetic cleansing preparations comprise 0 to 1.5% wt % of uncharged organic molecules having a molar mass <250 g/mol, based on the weight of the cleansing preparation. The perfume ingredient(s), especially selected from alpha-isomethyl ionone, benzyl alcohol, butylphenyl methylpropional, citronellol, coumarin, geraniol, limonene and linalool, present individually or in combination, together with their solvents, are included in the stated uncharged organic molecules having a molar mass <250 g/mol. Examples of further uncharged organic molecules having a molar mass <250 g/mol, which additionally have an aromatic ring system, are phenoxyethanol (molar mass 138.16 g/mol) and parabens, such as ethylparaben (166.15 g/mol) or propyl paraben (180.2 g/mol). It was possible to show that products according to the example formulations 2 and 13, in the production of which, in the former case, the incorporation of specific uncharged organic molecules having a molar mass <250 g/mol, namely parabens and phenoxyethanol, was dispensed with and, in the latter case, such molecules were incorporated, react differently with the packaging unit. If products according to example formulation 2, which comprise none of the stated uncharged organic molecules, are filled into packaging with an inner coating and stored for 4 months at 40° C., the inner coating remains intact (see FIG. 1, lower portion). In the case of products according to example formulation 13, which comprise such molecules, the inner coating is attacked; see in this regard FIG. 1, upper portion. Limited amounts of uncharged organic molecules having a molar mass <250 g/mol can be tolerated in the inventive cosmetic cleansing preparations, such as for example the above-described perfume ingredients, as long as the amount of uncharged organic molecules having a molar mass <250 g/mol does not exceed a value of 1.5 wt % based on the total weight of the preparation. The uncharged organic molecules having a molar mass <250 g/mol may also appear in the preparations via impurities and/or additions in the raw materials used. It is however more advantageous according to the invention, if 0 to 1.2 wt %, particularly preferably 0 to 1.0 wt %, of uncharged organic molecules having a molar mass <250 g/mol are present, in each case based on the weight of the cleansing preparations. These stated amounts include the amount of perfume ingredients and the solvents thereof. Preferred inventive cleansing preparations contain no parabens and no phenoxyethanol. Further preferred inventive cleansing preparations contain <0.05 wt % NaCl, based on the weight of the cleansing preparation, and no parabens and no phenoxyethanol.

The molar mass M of a substance is the quotient of the mass and the molar amount. The molar mass is described by the formula

M=m/n=N_{A}×m_{M}

where m is mass, n is molar amount, N_{A} is the Avogadro constant and m_{M} is molecular mass. If the empirical formula of a chemical compound is known, the molar mass can be calculated. For complex organic molecules, there are various methods for determining the molar mass, for example mass spectrometry.

Further, it is essential to the invention that the cleansing preparations comprise at most 0.5 wt % of NaCI based on the weight of the cleansing preparation. It is preferred that NaCI is present at <0.05 wt % based on the weight of the cleansing preparation. No NaCl is added to the preparations. Traces of NaCl contained in the cleansing preparations may originate from formulation constituents and/or the premixes thereof.

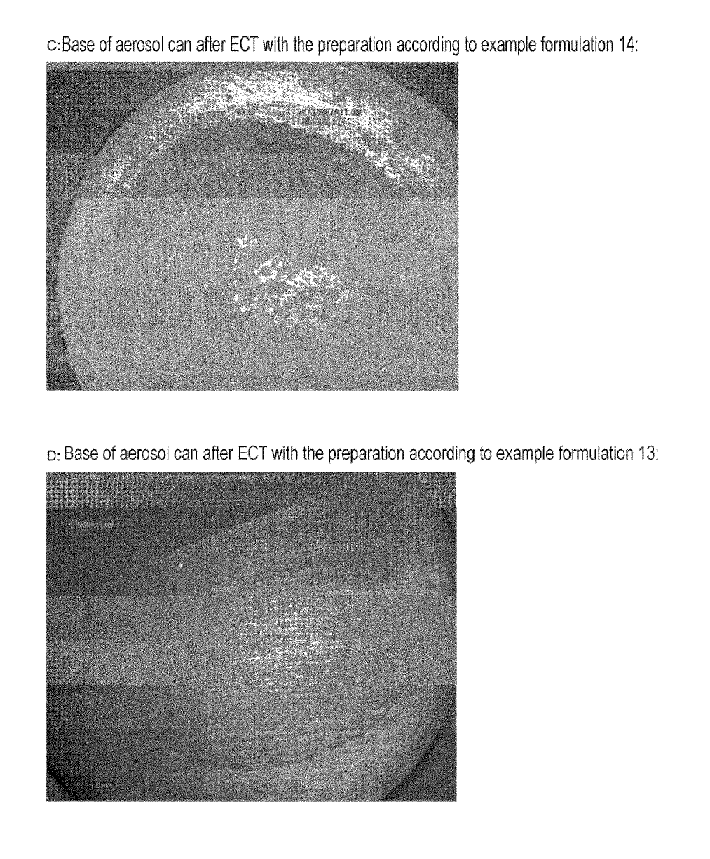

The strict limiting of the amount of uncharged organic molecules having a molar mass <250 g/mol in the cosmetic cleansing preparations and the substantial doing away with NaCl in the cleansing preparations makes it possible to prevent corrosion phenomena of the aerosol container: this is verified by what is referred to as an electrochemical corrosion test (ECT) and by electrochemical impedance spectroscopy (EIS). The methodology for ECT and EIS is described in the thesis by Marc Wilck, submitted on 2.3.2014. ECT is used to estimate what corrosion potentials arise in the case of a preparation that is decanted into a metal container. It makes it possible to ascertain whether an inner protective coating is required. Because EIS is carried out over a long period of time (2 weeks) with aerosol cans coated with inner protective coatings and preparations, it is possible to ascertain whether the investigated combinations of preparation, inner protective coating and aerosol can tend toward the formation of corrosion or not. For example, the results of ECT tests for the preparations according to example formulation 13 (chloride content 1.2 mg/kg) and 14 (chloride content 3900 mg/kg) are shown in FIG. 2. It becomes clear that preparations with a high chloride content lead to corrosion of the aerosol container. Therefore, no NaCl is added to the preparations according to the present invention.

Likewise, it is preferable if the pH value of the cleansing preparations is adjusted to a largely skin-neutral pH value of 4.1 to 5.8.

According to the invention, substances are therefore selected that are commonly used in order to stably adjust, and hold stable, the pH value in cosmetic preparations. Advantageously, these substances may be selected from the group of citric acid and lactic acid.

Since substances that are frequently used for the preservation of cosmetic preparations, for example phenoxyethanol and parabens, are in the group of uncharged organic molecules having a molar mass <250 g/mol, such substances cannot be used for preserving the inventive preparations. The inventive cosmetic cleansing preparations are therefore preserved with benzoic acid and/or a salt of benzoic acid, with salicylic acid and/or a salt of salicylic acid, and/or with sorbic acid and/or a salt of sorbic acid. Preservation with sodium benzoate is preferred. The inventive preservative substances are present at contents of 0.1 to 1.0 wt %, preferably 0.3 to 0.6 wt %, based on the weight of the cleansing preparation.

The inventive cleansing preparations are especially suitable for forming shower foams.

Another subject of the present invention is a method for preventing corrosion of aerosol containers, especially aerosol containers made of aluminum, coated with an inner coating, especially coated with epoxy phenolic coating, which is characterized in that said aerosol containers are filled with cleansing preparations,
which comprise ≤0.5 wt % of NaCI, based on the weight of the cleansing preparation, and
comprise 0 to 1.5% wt % of uncharged organic molecules having a molar mass <250 g/mol, based on the weight of the cleansing preparation,

and filled with propellant.

It is preferable if the NaCl content is <0.05 wt %. It is further preferable if the uncharged organic molecules having a molar mass <250 g/mol are present at a content of 0 to 1.2 wt %, preferably 0 to 1.0 wt %. The abovementioned stated amounts relate in each case to the weight of the cleansing preparation.

It is likewise preferable if NaCl is present at a content of <0.05 wt % and the uncharged organic molecules having a molar mass <250 g/mol are present at a content of 0 to 0.1 wt %, preferably 0 to 0.01 wt %. The abovementioned stated amounts relate in each case to the weight of the cleansing preparation.

Perfumes generally consist of odorants and solvents, mainly alcohols or other suitable solvents, optionally in combination with water. Odorants are essential oils of plant or animal origin, and nowadays increasingly synthetic odorants. In the context of the present invention, the terms odorant and perfume ingredient are synonymous. Perfumes are generally present at contents of 0.3 to 1.2 wt %, preferably 0.5 to 1.0 wt %, based on the cleansing preparation.

Another subject of the present invention is a method for preventing corrosion of aerosol containers, especially aerosol containers made of aluminum, coated with an inner coating, especially coated with epoxy phenolic coating, which is characterized in that said aerosol containers are filled with cleansing preparations, comprising
≤0.5 wt % of NaCl, based on the weight of the cleansing preparation,
≤10 wt % of surfactants, based on the active content and the weight of the cleansing preparation, selected from the group of anionic, amphoteric and nonionic surfactants,
wherein the cosmetic cleansing preparations comprise 0 to 1.5 wt % of uncharged organic molecules having a molar mass of <250 g/mol, and

filled with propellant.

“Post-foaming” or “foamable” should be understood to mean preparations that form a foam, with it being preferable that the foam forms on exiting the nozzle. Foaming involves gas bubbles (randomly) distributed in one (or more) liquid phase(s).

In aerosols, liquid droplets are (randomly) distributed in a gaseous phase.

In the context of the present invention, in aerosol foams, the gaseous propellants are present in the cleansing preparation, which is in a liquid phase, with the liquid phase surrounding the gaseous phase(s).

The inventive preparations are preferably present as foamable preparations. The inventive preparations are particularly preferably present as aerosol foams.

Further anionic and/or amphoteric and/or nonionic surfactants may be used in the inventive preparations, with the proviso that they do not prevent the present object being solved.

The following compounds are included in the anionic surfactants:

Acyl amino acids (and salts thereof), such as
1. Acyl glutamates, for example sodium acyl glutamate, sodium cocoyl glutamate, di-TEA-palmitoyl aspartate and sodium caprylic/capric glutamate,
2. Acyl peptides, for example palmitoyl hydrolyzed milk protein, sodium cocoyl hydrolyzed soy protein, and sodium/potassium cocoyl hydrolyzed collagen,
3. Sarcosinates, for example myristoyl sarcosine, TEA-lauroyl sarcosinate, sodium lauroyl sarcosinate and sodium cocoyl sarcosinate,
4. Taurates, for example sodium lauroyl taurate and sodium methyl cocoyl taurate,
5. Acyl acetates, lauroyl lactylate, caproyl lactylate.

Carboxylic acids and derivatives, such as
1. Ester carboxylic acids, for example calcium stearoyl lactylate, laureth-6-citrate and sodium PEG-4-lauramide carboxylate,
2. Ether carboxylic acids, for example sodium laureth-13-carboxylate and sodium-PEG-6-cocamide carboxylate,

Phosphoric acid esters and salts, such as, for example, DEA-oleth-10-phosphate and dilaureth phosphate,

Sulfonic acids and salts, such as
1. Acyl isethionates, e.g. sodium/ammonium cocoyl isethionate, sodium lauryl methyl isethionates,
2. Alkylaryl sulfonates,
3. Alkyl sulfonates, for example sodium cocomonoglyceride sulfate, sodium C12-14 olefin sulfonate, sodium lauryl sulfoacetate and magnesium PEG-3 cocamide sulfate,
4. Further sulfosuccinates, for example dioctyl sodium sulfosuccinate, disodium lauryl sulfosuccinate, disodium undecylene amido-MEA-sulfosuccinate and PEG-5 lauryl citrate sulfosuccinate,

and also

Sulfuric acid esters, such as
1. Further alkyl ether sulfates having different degrees of ethoxylation and mixtures thereof, for example ammonium-, magnesium-, MIPA-, TIPA-laureth-X sulfate, sodium myreth-X sulfate, and sodium C12-13-pareth-X sulfate, with X=1-5 ethoxy groups.
2. Alkyl sulfates, for example sodium-, ammonium- and TEA-lauryl sulfate.

The following compounds are included in the amphoteric surfactants:
1. Further acyl/dialkyl ethylenediamines, for example disodium acyl amphodipropionate, sodium acyl amphohydroxylpropyl sulfonate and sodium acyl amphopropionate,
2. N-alkylamino acids, for example aminopropyl alkylglutamide, alkylaminopropionic acid, sodium alkylimidodipropionate and lauroamphocarboxyglycinate.
3. Sultaines, for example, lauryl hydroxysultaine.

The following compounds are included in the nonionic surfactants:
1. Amine oxides, such as cocamidopropylamine oxide,
2. Ethers, for example, ethoxylated/propoxylated alcohols, laureth-X where X=2 to 10, wherein X signifies ethoxy groups, ethoxylated/propoxylated esters, ethoxylated/propoxylated cholesterols, ethoxylated propoxylated lanolin, ethoxylated/propoxylated polysiloxanes.

It is also possible to incorporate natural oils into the inventive preparations, which are however only used in small amounts, i.e. amounts 5. 0.1 wt %, based on the weight of the cleansing preparation. For example, almond oil and/or sunflower oil may be used in these amounts. Natural oils are understood to mean triglycerides which are primarily obtained from plants but also from animals and which are liquid at room temperature.

Inventive preparations may comprise one or more polymeric structurants. The polymeric structurants may be selected from the group of natural, nature-based or synthetic polymers. Synthetic polymers are advantageously acrylate based, preferably acrylate based copolymers. Particular preference is given to using the compound acrylates copolymer.

Natural polymers are also similarly well-suited and particular preference is given to using xanthan gum. Furthermore, the combination of acrylates copolymer and xanthan gum should advantageously be used.

Xanthan gum is generally used in concentrations of 0.1 to 0.5 wt % based on the cleansing preparation. Acrylates copolymer is used in concentrations of 0.5 to 3 wt % based on the cleansing preparation.

Advantageous propellants according to the invention are propellant gases such as propane, n-butane and/or isobutane. In this case, mixtures of these gases are preferred according to the invention. Mixtures of propellant gases that are particularly advantageous according to the invention have the following composition:

Mixture A: 72% isobutane, 23% propane, 5% butane,

Mixture B: 79.4% isobutane, 15.3% propane, 5.3% butane,

Mixture C: 60% butane, 20% isobutane, 20% propane.

Advantageously in the context of the present invention, the proportion by weight of propellant is selected to be in the range from 1.0 to 15 wt %, especially 2.0 to 10.0 wt %, based on the total weight of contents and propellant. Contents should be understood as the inventive cleansing preparation. The contents is accordingly present at a proportion by weight from 99 to 85 wt %, especially 98 to 90 wt % in the total preparation. Total preparation should be understood as the cleansing preparation together with the propellant.

The inventive cleansing preparations are advantageously present in the form of foamable or post-foamable preparations which are dispensed from aerosol containers and foam up on exiting the nozzle. Aerosol containers that are advantageous according to the invention are spray devices having a filling of predominantly liquid cleansing preparations which are under the pressure of a propellant. Such containers may be fitted with valves of a very wide variety of designs, which valves enable foaming up when dispensing the contents.

Mainly cylindrical vessels made of metal (aluminum, tinplate, contents <1000 ml) are considered in the context of the invention as pressurized gas containers, with compressive strength and breaking strength, corrosion resistance, easy fillability, etc., but also esthetic considerations, handleability, printability, etc., playing a role in the choice thereof. The maximum permissible operating pressure for spray cans made of metal at 50° C. is 12 bar and the maximum fill volume at this temperature is approximately 90% of the total volume.

Cans made of tinplate or aluminum are particularly advantageous in the context of the present invention. For the purpose of corrosion protection, metal cans may be coated on the inside (coated with silver or gold), for which purpose all customary inner protective coatings are suitable. In the context of the present invention, polyester, epoxyphenol and polyamideimide coatings are preferred. Film laminates made of polyethylene (PE), polypropylene (PP) and/or polyethylene terephthalate (PET) on the inside of the cans are also advantageous, especially for cans made of tinplate.

The pressurized gas containers are usually single-part or two-part, but in most cases three-part, cylindrical, conical or differently shaped.

The inner construction of the spray cans and also the valve construction are many and varied, depending on the intended use and the physical nature of the contents—e.g. whether it is in the form of a two-phase or three-phase system—and can be determined by those skilled in the art by simple trial and error without exercising inventive skill. For suitable embodiments, reference may be made to the “Aerosol Technologie Handbuch der Aersosol-Verpackung” [“Aerosol Technology, Handbook of Aerosol Packaging”] (Wolfgang Tauscher, Melcher Verlag GmbH Heidelberg/München, 1996).

Valves which are advantageous according to the invention can be designed with or without riser tube. The individual components from which valves according to the invention are usually constructed preferably consist of the following materials:
Disk: tinplate: uncoated, gold- or clear-coated, film-laminated (PE, PP or PET) aluminum: uncoated, silver- or gold-coated, different coating variants, Stoner Mudge design.
Seal: natural or synthetic elastomers or thermoplastic (sleeve gaskets, film-laminated in PE or PP) internal and external seals, e.g. made of perbunan, buna, neoprene, butyl, CLB, LDPE, viton, EPDM, chlorobutyl, bro mobutyl and/or various compounds.
Cone: polyimide (PA), polyoxymethylene (POM), brass and also various special materials, standard bores (e.g.: 0.25 to 0.70 mm or 2×0.45 to 2×1.00 mm), various shaft diameters
Spring: metal, particularly preferably V2A, stainless steel; plastic and also elastomer.
Casing: standard and impact
   VPH bores, RPT bores or slit for overhead applications
   materials: e.g. polyacetal, PA, PE, POM and the like
Riser tube: plastic (polymer resin), e.g. PE, PP, PA or polycarbonate.

Advantageous spray heads in the context of the present invention are, for example, foaming heads for upright use (holding can vertically) or foam heads for overhead application having one or more channels.

It is preferable according to the invention if the aerosol container is an aluminum can, which is coated on the inside with a protective coating, especially with epoxyphenol coating.

Optionally, when required, additives and auxiliaries customary in cosmetics may be incorporated into the preparations, such as, e.g. deodorizing substances, antiperspirants, insect repellents, vitamins, dyes, pigments which have a coloring action, flavorings, denaturing agents, plasticizers, moisturizers and/or humectants, antioxidants, UV screening agents, sensory additives, film-forming agents, active ingredients or other customary constituents of a cosmetic formulation, such as foam stabilizers or silicone derivatives, with the proviso that the additives and auxiliaries do not prevent the stated object from being solved.

By way of example, two inventive preparations were investigated regarding their mildness; the results can be found in the table below:

| | |
|---|---|
| 13 | L/D = 2.68 (moderately irritating) |
| 15 | L/D = 2.16 (moderately irritating) |

An RBC assay was carried out to determine the mildness.

The standard RBC assay (10 minutes' incubation) serves to estimate the in vivo eye irritancy potentials of surfactants and surfactant-containing products.
1. Hemolysis:
   A defined aliquot of isolated calf erythrocytes is incubated for 10 minutes with shaking at room temperature (RT) with a series of increasing concentrations of the WAS test specimen to be investigated (stock solution with formulations 1:100 w/v or 0.1% active content in PBS for raw materials). After centrifugation, the resulting supernatants are analyzed photometrically at 530 nm for their liberated hemoglobin (HbO₂) content. The relative degree of hemolysis is calculated therefrom and the parameter H50 [μl/ml] is determined from the concentration-response curve. This indicates the concentration of the test specimen at which 50% of hemoglobin is liberated.
2. HbO₂ denaturation:
   A defined aliquot of isolated calf erythrocytes is incubated for 10 minutes with shaking at RT with a fixed concentration of test specimen (1% w/v or 0.1% active content) and then centrifuged. The change in spectral absorption at 575 nm and 540 nm is measured compared to native HbO₂. The denaturation index DI [%] is calculated from the ratio of the absorption values to each other. Na lauryl sulfate serves as 100% standard (0.1% active content).
3. LID quotient:
   The quotient is the ratio of the hemolysis parameters (H50) and denaturation (DI) and is used to characterize and classify the test specimens investigated.

The methodology is explicitly described in Mol. Toxicol. 1(4), 1987-1988, page 525 et seq.

The results show that both preparations only have a moderate irritancy potential for the eye mucous membrane. The preparation according to example formulation 15 is the inventive preparation without parabens and phenoxyethanol. The product according to this preparation performs better in the abovementioned test than the comparative product. Since this test relates to determining the irritancy potential for the eye mucous membrane, the values obtained are also an indicator of the overall mildness of products, with products having a moderate irritancy potential for the eye mucous membrane being able to be classified overall as mild products.

Products according to example formulations 14 and 13 were subjected to an ECT, and the results are shown in FIG. 2. The results of the ECT show passivation for the combination of preparation according to example formulation 13 and aluminum can. For the preparation according to example formulation 14, the results and the image show pit corrosion. The difference between the two preparations essentially lies in the chloride concentration. The preparation according to example formulation 13 essentially comprises fewer chloride ions.

Using EIS, an inner protective coating for a product according to example formulation 16 in an aluminum can could be identified, which counteracts corrosion of the aerosol container. The coating identified was an epoxyphenol coating.

FIG. 1 shows two aerosol containers cut open. In the upper portion it can be seen that the inner protective coating of epoxyphenol is detaching from the aerosol can made of aluminum. The cleansing preparation that was filled into this can contained 1.6 wt % of uncharged organic molecules having a molar mass <250 g/mol, in the form of methylparaben, ethylparaben and phenoxylethanol. The cleansing preparation was stored for 4 months at 40° C. in the aerosol can.

The lower portion of the figure shows an aerosol can that was filled with a cleansing preparation that did not contain any methylparaben, ethylparaben or phenoxylethanol. The inner protective coating was not attacked. Hence, the negative effects of methylparaben, ethylparaben and phenoxylethanol on the resistance of the inner coating have been demonstrated. Once the inner protective coating has been damaged, corrosion of the aerosol container begins.

### EXAMPLES

Example formulations of cleansing preparations without propellant:

| | Example No. | | | | | |
|---|---|---|---|---|---|---|
| | 1 | 2 | 3 | 4 | 5 | 6 |
| Sodium Laureth Sulfate | 3 | 3.8 | 4 | 4.5 | 5 | 4 |
| Disodium Laureth Sulfosuccinate | 3 | 4.9 | | 2.2 | 3 | |
| Decylglycoside | | | 5 | | | 4 |
| Cocoglycoside | 2 | | | | | |
| Sodium acyl glutamate | | | | | | |
| Disodium cocoyl glutamate | | | | 1 | | 1 |
| PEG-200 Hydrogenated Glyceryl | 1 | 0.5 | 0.1 | | | |
| Palmate | | | | | | |
| PEG-14 M | | 0.2 | | 0.1 | 0.4 | |
| PEG-90 M | 0.2 | | 0.4 | | | |
| Acrylates Copolymer | 1 | | | 3 | | |
| Perfume | 1 | 0.8 | 1 | 1.2 | 1 | 0.8 |
| PEG-40 Hydrogenated Castor Oil | 0.7 | 0.5 | 0.7 | 0.9 | 0.6 | 0.6 |
| Glycerol | 2 | 1 | | 2 | | 3 |
| Citric Acid | 0.6 | 0.8 | 0.7 | 0.5 | 0.8 | 0.4 |
| Sodium Benzoate | 0.5 | 0.45 | 0.45 | 0.4 | 0.3 | 0.5 |
| Xanthan Gum | 0.4 | 0.3 | 0.2 | 0.1 | | |
| Sunflower oil or almond oil | 0.1 | | | 0.05 | | |
| Water | to 100 | to 100 | to 100 | to 100 | to 100 | to 100 |
| | Example No. | | | | | |
| | 7 | 8 | 9 | 10 | 11 | 12 |
| Sodium Laureth Sulfate | | | 2 | | | |
| Disodium Laureth Sulfosuccinate | 2 | | | | 3 | |
| Decylglycoside | | 4 | 3 | | | |
| Cocoglycoside | | | | 3.3 | 3 | 5 |
| Sodium acyl glutamate | 4 | | 4 | 5.9 | 3 | 3 |
| Disodium cocoyl glutamate | | 4 | | | | |
| Cocamidopropyl betaine | | | | | | |
| PEG-200 Hydrogenated Glyceryl | | | | 1 | 0.5 | 0.1 |
| Palmate | | | | | | |
| PEG-14 M | | 0.2 | | 0.1 | 0.4 | |
| PEG-90 M | 0.2 | | 0.4 | | | |
| Acrylates Copolymer | 1 | | 3 | | | |
| Perfume | 1 | 0.8 | 1 | 1.2 | 1 | 0.8 |
| PEG-40 Hydrogenated Castor Oil | 0.7 | 0.5 | 0.7 | 0.9 | 0.6 | 0.6 |
| Glycerol | 2 | 1 | | 2 | | 3 |
| Citric Acid | 0.6 | 0.8 | 0.7 | 0.5 | 0.8 | 0.4 |
| Sodium Benzoate | 0.5 | 0.45 | 0.45 | 0.4 | 0.3 | 0.5 |
| Ethylparaben | | | | | | |
| Methylparaben | | | | | | |
| Phenoxyethanol | | | | | | |
| Xanthan Gum | 0.4 | 0.3 | 0.2 | 0.1 | | |
| Sunflower oil or almond oil | 0.1 | | | 0.05 | | |
| Polyquaternium-7 | | | | | | |
| Water | to 100 | to 100 | to 100 | to 100 | to 100 | to 100 |

Example No.

| | 13 | 14 | 15 | 16 |
|---|---|---|---|---|
| Sodium Laureth Sulfate | 3.7 | 3.5 | 3.3 | 4 |
| Disodium Laureth Sulfosuccinate | 3.5 | | 3.3 | 3.5 |
| Decylglycoside | | | | |
| Cocoglycoside | | | | |
| Sodium acyl glutamate | | | | |
| Disodium cocoyl glutamate | | | | |
| Cocamidopropyl betaine | | 3.5 | | |
| PEG-200 Hydrogenated Glyceryl | | | | |
| Palmate | | | | |
| PEG-14 M | 0.1 | | 0.2 | 0.1 |
| PEG-90 M | | | | |
| Acrylates Copolymer | | | | |
| Perfume | 0.8 | 1 | 0.9 | 0.8 |
| PEG-40 Hydrogenated Castor Oil | 0.5 | 0.7 | 0.5 | 0.8 |
| Glycerol | 2.5 | 1 | 2 | 1.5 |
| Citric Acid | | | 0.6 | 0.75 |
| Sodium Benzoate | | | 0.5 | 0.35 |
| Ethylparaben | 0.4 | 0.3 | | |
| Methylparaben | 0.4 | 0.3 | | |
| Phenoxyethanol | 0.9 | 0.8 | | |
| Xanthan Gum | 0.5 | | 0.5 | 0.1 |
| Sunflower oil or almond oil | 0.05 | 0.01 | 0.1 | 0.01 |
| Polyquaternium-7 | | 0.3 | | |
| Water | to 100 | to 100 | to 100 | to 100 |

1.-20. (canceled)

21. A cosmetic cleansing preparation, wherein the preparation is present, together with a propellant, in an aerosol container with an inner protective coating, and comprises, based on a total weight of the cleansing preparation,
≤0.5 wt % NaCl,
≤10 wt % of one or more surfactants selected from anionic, amphoteric and nonionic surfactants, and
0 to 1.5 wt % of uncharged organic molecules having a molar mass of <250 g/mol.

22. The preparation of claim 21, wherein the propellant is a propellant gas or a mixture of propellant gases selected from propane, n-butane and isobutane.

23. The preparation of claim 21, wherein the propellant is present in a concentration of from 1.0 to 15 wt %, based on the total weight of the preparation.

24. The preparation of claim 21, wherein the aerosol container is an aluminum can.

25. The preparation of claim 21, wherein the protective coating is an epoxyphenolic coating.

26. The preparation of claim 24, wherein the protective coating is an epoxyphenolic coating.

27. The preparation of claim 21, wherein the preparation is free of soaps.

28. The preparation of claim 21, wherein the one or more surfactants are present in a concentration of from 1 to 10 wt %.

29. The preparation of claim 21, wherein the one or more surfactants comprise one or more anionic surfactants selected from sodium laureth sulfate, disodium laureth sulfosuccinate, and sodium cocosulfate.

30. The preparation of claim 21, wherein the one or more surfactants comprise only one or more anionic surfactants.

31. The preparation of claim 21, wherein the one or more surfactants comprise one or more nonionic surfactants selected from alkyl polyglycosides and PEG-200 hydrogenated palmate.

32. The preparation of claim 31, wherein the alkyl polyglycosides are selected from decyl glycoside and coco glycoside.

33. The preparation of claim 21, wherein the preparation comprises not more than 1.2 wt % of uncharged organic molecules having a molar mass of <250 g/mol.

34. The preparation of claim 21, wherein the cleansing preparation is free of parabens and phenoxyethanol.

35. The preparation of claim 21, wherein the preparation comprises one or more preservative substances selected from benzoic acid and salts thereof, salicylic acid and salts thereof, sorbic acid and salts thereof.

36. The preparation of claim 21, wherein the preparation comprises from 0.1 to 1.0 wt % of one or more preservative substances.

37. The preparation of claim 21, wherein the preparation comprises <0.05 wt % of NaCl.

38. The preparation of claim 21, wherein the preparation has a pH value of from 4.1 to 5.8.

39. A method of preventing corrosion of an aerosol container filled with a cleansing preparation, wherein the method comprises filling the aerosol container with a propellant and a cleansing preparation which comprises, based on a total weight of the cleansing preparation,
≤0.5 wt % of NaCl, and
0 to 1.5% wt % of uncharged organic molecules having a molar mass <250 g/mol.

40. A method of preventing corrosion of an aerosol container, wherein the method comprises filling the aerosol container with a propellant and the cleansing preparation of claim 1.
